(19) United States
(12) Patent Application Publication
(10) Pub. No.: US 2021/0324001 A1
(43) Pub. Date: Oct. 21, 2021

(54) NEW PURIFICATION METHOD

(71) Applicant: MERCK BIODEVELOPMENT S.A.S., MARTILLAC (FR)

(72) Inventors: CHRISTELLE SIHABOUT, FLOIRAC (FR); DAVID DELVAILLE, PESSAC (FR)

(21) Appl. No.: 17/271,615

(22) PCT Filed: Aug. 29, 2019
(86) PCT No.: PCT/EP2019/073140

§ 371 Date: Feb. 26, 2021

(30) Foreign Application Priority Data
Aug. 29, 2018 (EP) 18306146.4

(51) Int. Cl. C07K 1/36; C07K 1/18; C07K 1/34

(52) U.S. Cl. CPC C07K 1/36; C07K 1/34; C07K 1/18

(57) ABSTRACT

Methods of purifying a peptide are provided. Also provided is an improved method of purifying a polypeptide of interest from a sample containing the polypeptide of interest and impurities in which clarification and the first purification step are part of one step only.

### FIELD OF INVENTION

The present invention relates to method for purifying a polypeptide. The present invention more particularly relates to the improved purification a polypeptide of interest from a sample containing said polypeptide of interest and impurities. In said improved method the clarification and the first purification step are part of one step only.

### BACKGROUND OF THE INVENTION

In a general way, a manufacturing process to obtain a drug substance, such as a polypeptide, in biotechnology is separated in several steps. Firstly, the host cell expressing the molecule of interest is produced in large quantity with a fermenter (microbial process) or a bioreactor (mammalian process). At the end of the culture step, the molecule of interest is harvested, this step is either by centrifugation or by filtration.

If the molecule of interest is insoluble (essentially for microbial processes), refolding step must be performed before to obtain soluble forms.

A clarified product is obtained, and the next step is a chromatography technique to capture the molecule and removed some contaminants. This step is called capture. An additional chromatography step is always necessary to refine the molecule, it is the polishing step followed by an ultrafiltration to concentrate the molecule of interest and a diafiltration step to formulate the product in specified conditions. For instance, WO9747650 describes a method of purification of a polypeptide involving clarification, followed by two ion exchange chromatography steps or WO0048703 proposes the use of at least one cross flow filtration, following the clarification step, to purify a polypeptide.

There is a need for further purification methods in order to improve the timing and the costs of said purification steps, which are usually time consuming and very expensive.

### SUMMARY OF THE INVENTION

As described herein, the present invention is related to a method for purifying a polypeptide of interest from a sample containing said polypeptide of interest and impurities, said process comprising the steps of: i) contacting the sample containing the polypeptide of interest and impurities with a chromatography resin, without submitting the sample to an initial clarification step; ii) incubating the sample from step i) with the chromatography resin for a sufficient time to allow the resin to bind the polypeptide of interest, preferably under stirring conditions; iii) recirculating the chromatography resin in hollow fibres or any tangential filtration system, with or without concentrating the polypeptide of interest in order to obtain less volume; iv) washing by diafiltration the sample containing the polypeptide of interest and the impurities in order to remove impurities; v) eluting the polypeptide of interest from the chromatography resin; and vi) recovering the purified polypeptide of interest from the chromatography resin by diafiltration.

The chromatography resin to be used according to the present invention can be selected from the group consisting of protein A, protein A related, cation-exchange, anion-exchange or mixed-mode resins.

The sample containing said polypeptide of interest and impurities, to be purified according to the present invention, is preferably an harvest fluid from a cell culture or a cell culture, either a crude harvest fluid or crude cell culture (for instance when the polypeptide of interest has been secreted) or an harvest fluid or a cell culture that has been submitted to lysis, solubilization and refolding (for instance when the polypeptide has been produced internally, in the cytoplasm or periplasm of a cell, either soluble or in inclusion bodies).

The polypeptide of interest according to the present invention has been produced in a recombinant host and is either secreted by the recombinant host or is contained inside cytoplasm or periplasm of the recombinant host. Preferably, the recombinant host is a prokaryotic cell such as a bacterium or lower eukaryotic such as yeast. In a prefer embodiment the polypeptide of interest is selected from the group consisting of a recombinant protein, a fusion protein, an immunoglobulin or an antibody, or any fragments thereof.

### Definitions

The term “buffer” is used according to the art. An “equilibration buffer” is a buffer used to prepare the chromatography resin to receive the sample to be purified. A “loading buffer” refers to the buffer used to load the sample on the chromatography column or on a filter. A “wash buffer” is a buffer used to wash the resin. Depending on the mode of the chromatography it will allow the removal of the impurities (in bind/elute mode) or the collection of the purified sample (in flowthrough mode). An “elution buffer” refers to the buffer that is used to unbind the sample from the chromatographic material. This is possible thanks to the change of the chemical properties of the buffers (e.g. ionic strength and/or pH) between the load/wash buffers and the elution buffer. The purified sample containing the polypeptide of interest will thus be collected as an eluate.

The term “resin” or “chromatographic material” refer to any solid phase allowing the separation of the polypeptide to be purified from the impurities. Said resin or chromatographic material may be an affinity, an anionic, a cationic or a mixed mode resin/chromatographic material. The resins according to the invention should be spherical shape beads-based resins.

The term tangential flow filtration (also referred to as tangential filtration or cross flow filtration) is a technique which uses a pump to circulate a sample across the surface of a membrane (“tangential” to the membrane surface). The applied transmembrane pressure acts as the driving force to transport solute and small molecules through the membrane. The cross flow of liquid over the membrane surface sweeps retaining molecules from the surface, keeping them in the circulation stream.

The term “tangential filtration system” refers to a device allowing to perform tangential flow filtration. Such a device can be for instance a capsule, a cassette or a hollow fibre module. A cassette for tangential flow filtration is a set-up of membranes layer housed in a multilevel structure. A membrane layer consists of three main components which are the channel spacer (which disperses the sample across the membrane surface), the membrane and a support. The separation of the molecules and particles is in function of their size. A cassette of tangential flow filtration varies as a function of their material, cut-off threshold and area membrane. The main suppliers are Merck Millipore, GE Healthcare, Sartorius, Pall and Spectrum.

The term “hollow fibre” refers to a class of membranes comprising a semi-permeable barrier. They can be used to clarify high viscosity products such as fermenters harvest. The hollow fibres are assembled in parallel forming a module. An industrial module can have a several thousand fibres. The separation of the molecules and particles is in function of their size. A module of hollow fibre varies as a function of their material, cut-off threshold, area membrane, lumen pore-size and their length. The main suppliers are GE Healthcare and Spectrum (who have developed modified PES (mPES) to improve the filtration).

The term “clarification” as used herein refers to the step of removal of hosts and host debris to enable product capture on a chromatographic column. Commonly, clarification is performed via centrifugation and/or filtration, such as microfiltration, depth filtration or yet tangential flow filtration (TFF).

The term “polypeptides” as used herein also includes peptides and proteins and refers to compound comprising two or more amino acid residues. The term includes but is not limited to, a cytokine, a growth factor (such as fibroblast growth factors), a hormone, a fusion protein, an antibody or a fragment thereof. A therapeutic protein refers to a protein that can be used or that is used in therapy. The term “protein” or “polypeptide” are herein used interchangeably.

The term “recombinant polypeptide” (also referred to as recombinant protein) means a protein produced by recombinant technics. Recombinant technics are well within the knowledge of the skilled person (see for instance Sambrook et al., 1989, and updates).

The term “Fc fusion protein” encompasses the combination (also called fusion) of at least two proteins or at least two proteins fragments to obtain one single protein, including at least an Fc portion, such as an antibody moiety.

The term “antibody”, and its plural form “antibodies”, includes, inter alia, polyclonal antibodies, affinity-purified polyclonal antibodies, monoclonal antibodies, and antigen-binding fragments. Antibodies are also known as immunoglobulins. Genetically engineered antibodies are called recombinant antibodies. Recombinant intact antibodies or fragments, such as chimeric antibodies, humanised antibodies, human, fully human antibodies, as well as synthetic antigen-binding peptides and polypeptides, such as nanobodies, scFv or Fab are also included. Also encompassed are SEEDbodies. The term SEEDbody (SEED for Strand-Exchange Engineered Domain; plural form: SEEDbodies), refers to a particular type of antibody comprising derivative of human IgG and IgA CH3 domains, creating complementary human SEED CH3 heterodimers that are composed of alternating segments of human IgG and IgA CH3 sequences. They are asymmetric fusion proteins. SEEDbodies and the SEED technology are described in Davis et al. 2010 or U.S. Pat. No. 8,871,912 which are incorporated herein in their entirety.

Units, prefixes and symbols are used according to the standards (International System of Units (SI)).

### DETAILED DESCRIPTION OF THE INVENTION

There is a need for further purification methods in order to improve the duration and the costs of said steps, which are usually time consuming and very expensive. The present invention is based on the finding from the inventors that it was possible to improve the duration and the costs of purification methods in combining the clarification step with the first chromatography step, in a new one-step called clapture. As shown in the example section, with the method according to the present invention, it was possible to reduce by a factor 3 the time of clarification/first chromatography and it was possible to reduce by at least 65% (for 1 run) the costs linked to these steps. Advantages of this one-step procedure are for instance: no need to pack large chromatography column, less water consumption (no cleaning in place for chromatography skid), time saving due to steps eliminated (such as sanitization, storage), etc. According to the invention, the resin is just added into the sample (such as crude harvest) and the entire mix is filtrated by hollow-fibres. Contaminants are removed, and product of interest is recover as after a chromatography capture step.

Therefore, in a first aspect, the present invention provides a method for purifying a polypeptide of interest from a sample containing said polypeptide of interest and impurities, said process comprising the steps of:
i) contacting the sample containing the polypeptide of interest and impurities with a chromatography resin, without submitting the sample to an initial clarification step;
ii) incubating the sample from step i) with the chromatography resin for a sufficient time to allow the resin to bind the polypeptide of interest, preferably under stirring conditions;
iii) recirculating the chromatography resin in hollow fibres or any tangential filtration system, with or without concentrating the polypeptide of interest in order to obtain less volume;
iv) washing by diafiltration the sample containing the polypeptide of interest and the impurities in order to remove impurities;
v) eluting the polypeptide of interest from the chromatography resin; and
vi) recovering the purified polypeptide of interest from the chromatography resin by diafiltration.

In a second aspect, the present invention provides a method for producing a polypeptide of interest comprising the step of culturing a recombinant host, recovering (or harvesting) all or part of the host cell culture (being defined as a sample containing the polypeptide of interest) and further comprising purifying said polypeptide of interest from said sample containing said polypeptide of interest and impurities, wherein the purification comprises the steps of:
i) contacting the sample containing the polypeptide of interest and the impurities with a chromatography resin, without submitting the sample to an initial clarification step;
ii) incubating the sample from step i) with the chromatography resin for a sufficient time to allow the resin to bind the polypeptide of interest, preferably under stirring conditions;
iii) recirculating the chromatography resin in hollow fibres or any tangential filtration system, thereby concentrating the polypeptide of interest while removing the impurities;
iv) washing by diafiltration the sample containing the polypeptide of interest and the impurities in order to remove impurities;
v) eluting the polypeptide of interest from the chromatography resin; and
vi) recovering the purified polypeptide of interest from the chromatography resin by diafiltration.

In the context of the present invention as a whole, the hollow fibre can be selected from the group consisting of (but not limited to) ReadyToProcess single-use hollow fibre cartridges, MidiKros, MiniKros or MicroKros modules. It is selected in function of their membrane composition, cut-off threshold, membrane area, lumen pore size and supplier. Examples of such hollow fibres are ReadyToProcess single-use hollow fibre cartridges and MidiKros modules, having a cut-off of 0.22 μm and a lumen of 1 mm. The membrane area depend of the volume to be filtrated (at small scale, the filterability to target is 200 L/m²).

In the context of the present invention as a whole, the chromatography resin can be selected from the group consisting of protein A, protein A related, cation-exchange, anion-exchange and mixed-mode. Should the preferred chromatography resin be a cation-exchange resin, said resin can be for instance selected from the group consisting of (but not limited to): SP-SFF, Eschmuno CPS, poros XS, poros 50HS, Fractogel SO₃⁻, GIGA Cap C650M or GIGA CAP S650M. This resin will be preferred in case of the purification of a protein having a pI above the pH of the sample in normal conditions. Should, the preferred chromatography resin be a protein A resin, said resin can be for instance selected from the group consisting of (but not limited to): MABSELECT™, MABSELECT™ SuRe, MABSELECT™ SuRe LX, AMSPHERE™ A3, TOYOPEARL AF-rProtein A-650F, TOYOPEARL AF-HC, PROSEP®-vA, PROSEP®-vA Ultra, PROSEP® Ultra Plus or ESHMUNO-A® and any combination thereof. Protein A can be one of the alternative material of choice for instance in case of purification of an Fc-protein or of an immunoglobulin. Should the preferred chromatography resin be an anion exchange resin, said resin can be for instance selected from the group consisting of (but not limited to): Q Sepharose FF, Capto Q Impres, Capto Q, Capto DEAE, Poros 50HQ, Poros XQ, Fractogel TMAE, Fractogel DMEA, Fractogel DEAE or Eshmuno Q. This resin will be preferred in case of the purification of a protein having a pI below the pH of the sample in normal conditions. Should the preferred chromatography resin be a mixed mode resin, said resin can be for instance selected from the group consisting of (but not limited to): MEP Hypercel or Capto Adhere.

The skilled person will understand that in order to bind to the resin, certain conditions of pH and salt of the sample to be purified have to be met (loading conditions), as a function of the protein to be purified and of the resin that is used for the clapture step. In same cases, the sample has therefor to be adjusted (e.g. modification of its pH and/or of its conductivity). The skilled person would understand that, in the context of the present invention as a whole, should the resin be a cation exchange resin, the pH of the sample containing the protein of intested has to be lower than the pI of the protein of interest. Said pH is preferably at least 1 unit pH lower than the pI of the protein to maximize the efficiency of the resin. As an example, should the pI of the protein of interest be of 10.0, a proper range of pH for the sample to be purified would be preferably 6.5.0 to 9.0, such as 7.0, 7.5, 8, 8.5, or 9.0. Alternatively, the skilled person would understand that, in the context of the present invention as a whole, should the resin be an anion exchange resin, the pH of the sample containing the protein of intested has to be higher than the pI of the protein of interest. Said pH is preferably at least 1 pH higher than the pI of the protein to maximize the efficiency of the resin. As an example, should the pI of the protein of interest be of 5.5, a proper range of pH for the sample to be purified would be preferably 6.5 to 8.5, such as 6.5, 7.0, 7.5, 8.0 or 8.5. The skilled person will know from the common general knowledge how to adapt the pH of the sample to the resin that is used, whatever the type of resin that is used (e.g. protein A, protein A related, mixed-mode and hydrophobic interaction chromatography resins).

The skilled person will understand that in order to equilibrate the resin, certain conditions of pH and salt of the equilibration buffer have to be met, for the purpose that the resin binds the protein of interest. The skilled person would understand that, in the context of the present invention as a whole and depending on the resin he has chosen to use, he can vary the pH and/or the conductivity of the solution thanks to the properties of the equilibration buffer. For instance, should the resin be a cation exchange resin, he can use an equilibration buffer having a pH lower than the pI of the protein of interest. Said pH is preferably 1-unit pH lower than the pI of the protein to maximize the efficiency of the resin. As an example, should the pI of the protein of interest be of 10.0, a proper range of pH for the elution buffer would be preferably 7.0 to 9.0, such as 7.0, 7.5, 8.0, 8.5 or 9.0. Alternatively, the skilled person would understand that, should the resin be an anion exchange resin, he can use an equilibration buffer having a pH higher than the pI of the protein of interest. Said pH is preferably 1-unit pH higher than the pI of the protein to maximize the efficiency of the resin. As an example, should the pI of the protein of interest be of 5.5, a proper range of pH for the equilibration buffer would be preferably 6.5 to 8.5, such as 6.5, 7.0, 7.5, 8.0 or 8.5. In another alternative, whatever the type of resin that is used, the skilled one can use an equilibration buffer preferably with low conductivity, such as an equilibration buffer comprising below 0.2M of salt, preferably below 0.15 M. The equilibration buffer has for instance a salt content in a range of 0 to 0.12 M, such as 0, 0.01, 0.02, 0.03, 0.04, 0.05, 0.06, 0.07, 0.08, 0.09, 0.10, 0.11, 0.12M. Preferably the equilibration buffer has a conductivity in the range of about 1 to about 20 mS/cm, even preferably 2 to about 20 mS/cm, such as 2, 3, 4, 5, 6, 7, 8, 9, 10, 11, 12, 13, 14, 15, 16, 17, 18, 19 or 20 mS/cm. Preferably the salt that is used to have a low conductivity equilibration buffer is selected from the group consisting of (but not limited to) NaCl or ammonium sulphate. Said equilibration buffer can consist of various species such as (but not limited to) phosphate, citrate, acetate, TRIS.

The skilled person will understand that in order to wash the resin, certain conditions of pH and salt of the washing buffer have to be met, for the purpose that the impurities must be removed and separate of the protein of interest. The skilled person would understand that, in the context of the present invention as a whole, he can vary the pH and/or the conductivity of the solution thanks to the properties of the washing buffer. For instance, should the resin be a cation exchange resin, he can use a washing buffer having a pH lower than the pI of the protein of interest. Said pH is preferably 1-unit pH lower than the pI of the protein to maximize the efficiency of the resin. As an example, should the pI of the protein of interest be of 10.0, a proper range of pH for the washing buffer would be preferably 7.0 to 9.0, such as 7.0, 7.5, 8.0, 8.5 or 9.0. Alternatively, the skilled person would understand that, should the resin be an anion exchange resin, he can use a washing buffer having a pH higher than the pI of the protein of interest. Said pH is preferably 1-unit pH higher than the pI of the protein to maximize the efficiency of the resin. As an example, should the pI of the protein of interest be of 5.5, a proper range of pH for the washing buffer would be preferably 6.5 to 8.5, such as 6.5, 7.0, 7.5, 8.0, 8.5. In another alternative, whatever the type of resin that is used, the skilled one can use a washing buffer with low conductivity, such as a washing buffer comprising below 0.2M of salt, preferably below 0.15 M. The washing buffer has for instance a salt content in a range of 0 to 0.12 M, such as 0, 0.01, 0.02, 0.03, 0.04, 0.05, 0.06, 0.07, 0.08, 0.09, 0.10, 0.11 or 0.12M. Preferably the washing buffer has a conductivity in the range of about 1 to about 20 mS/cm, even preferably 2 to about 20 mS/cm, such as 2, 3, 4, 5, 6, 7, 8, 9, 10, 11, 12, 13, 14, 15, 16, 17, 18, 19 or 20 mS/cm. Preferably the salt that is used to have a low conductivity washing buffer is selected from the group consisting of (but not limited to) NaCl or ammonium sulphate. Said washing buffer can consist of various species such as (but not limited to) phosphate, citrate, acetate, TRIS.

The skilled person would understand that, in the context of the present invention as a whole, a second wash after the previous one can be applied in order to remove more impurities. The skilled person would understand that, he can vary the pH and/or the conductivity of the solution thanks to the properties of the washing buffer. For instance, should the resin be a cation exchange resin, he can use a second washing buffer having a pH higher than the pH of the first wash buffer but lower than pH of elution buffer. Alternatively, the skilled person would understand that, should the resin be an anion exchange resin, he can use a second washing buffer having a pH lower than the pH of the first wash buffer but higher than pH of elution buffer. In another alternative, whatever the type of resin that is used, the skilled one can use a second washing buffer with a conductivity higher than the first wash buffer but lower than the elution buffer.

Similarly, the skilled person will understand that in order to elute the protein of interest from the resin, certain conditions of pH and salt of the elution buffer have to be met, as a function of the protein to be purified and of the resin that is used for the clapture step. The skilled person would understand that, in the context of the present invention as a whole, he can vary the pH and/or the conductivity of the solution thanks to the properties of the elution buffer. For instance, should the resin be a cation exchange resin, he can use an elution buffer having a pH higher than the pI of the protein of interest.

Said pH is preferably 1-unit pH higher than the pI of the protein to maximize the efficiency of the resin. As an example, should the pI of the protein of interest be of 10.0, a proper range of pH for the elution buffer would be preferably 11.0-12.0, such as 11.0, 11.1, 11.2, 11.3, 11.4, 11.5, 11.6, 11.7, 11.8, 11.9 or 12.0. Alternatively, the skilled person would understand that, should the resin be an anion exchange resin, he can use an elution buffer having a pH lower than the pI of the protein of interest. Said pH is preferably 1-unit pH lower than the pI of the protein to maximize the efficiency of the resin. As an example, should the pI of the protein of interest be of 5.5, a proper range of pH for the elution buffer would be preferably 3.0-4.5, such as 3.0, 3.1, 3.2, 3.3, 3.4, 3.5, 3.6, 3.7, 3.8, 3.9, 4.0, 4.1, 4.2, 4.3, 4.4 or 4.5. In another alternative, whatever the type of resin that is used, the skilled one can use an elution buffer with high conductivity, such as an elution buffer comprising above 0.4M of salt, preferably, the elution buffer has a salt content in a range of 0.4 to 3 M, even preferably 0.5 to 2 M, such as 0.5, 0.6, 0.7, 0.8, 0.9, 1.0, 1.1, 1.2, 1.3, 1.4, 1.5, 1.7, 1.8, 1.9 or 2M. Preferably the elution buffer has a conductivity in the range of about 40 to about 300 mS/cm, even preferably 50 to about 200 mS/cm, such as 50, 60, 70, 80, 90, 100, 110, 120, 130, 140, 150, 170, 200 mS/cm. Preferably the salt that is used to have a high conductivity elution buffer is selected from the group consisting of (but not limited to) NaCl or Ammonium sulphate. Said elution buffer can consist of various species such as (but not limited) phosphate, citrate, Tris, acetate

In the context of the present invention as a whole, the sample containing the polypeptide of interest and impurities is selected from the group consisting of (but not limited to) a cell culture, a supernatant of cell culture or a harvest fluid from cell culture. Preferably said sample is either 1) a crude cell culture, a crude supernatant of cell culture or a crude harvest fluid, should the protein be secreted in the culture medium or 2) a crude cell culture, crude supernatant of cell culture, crude harvest fluid, crude cell homogenate submitted to lysis, solubilization and refolding, should the protein to be purified be in the form of inclusion bodies.

In the context of the present invention as a whole, the recombinant cell is a prokaryotic cell such as a bacterial cell or a lower eukaryotic cell such as a yeast. Should the prokaryotic cell be a bacterial cell it can be selected from the group consisting of (but not limited to) Gram-negative or Gram-positive bacteria, such as Escherichia coli (E. coli), Bacillus subtilis (B. subtilis), Lactobacillus, Lactococcus, Pseudomonas aeruginosa (P. aeruginosa), Salmonella typhimurium, or Serratia marcescens. Should the cell be a yeast it can be selected from the group consisting of (but not limited to), Saccharomyces cerevisiae or Pichia pastoris.

The polypeptide of interest (also herein referred to as protein of interest), in the context of the present invention as a whole, is selected from the group consisting of a recombinant protein, a fusion protein, an immunoglobulin or an antibody, or any fragments thereof as defined herein. It includes for instance (but not limited to) a cytokine, a growth factor (such as fibroblast growth factors), a hormone, a nanobody or a SEEDbody.

In the context of the present invention as a whole, the impurities to be removed are selected from at least one of the group consisting of aggregates or fragments of the polypeptide of interest, or mixtures thereof, of the protein of interest, one or more of host cell proteins, endotoxins, viruses, nucleic acid molecules, lipids, polysaccharides, and any combinations thereof.

In the context of the present invention as a whole, the purified polypeptide recovered from step v) is optionally further purified through at least one additional purification step. The at least one additional purification step can be selected from the group consisting of affinity chromatography, cation exchange chromatography, anion exchange chromatography and mixed mode chromatography. This optional additional purification step when it is performed, is called step vi). The purified polypeptide recovered from step v) and/or step vi) can be optionally further concentrated using any filtration system such as ultrafiltration (UF), diafiltration (DF) or a combination thereof (UF/DF).

Other embodiments of the invention within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims that follow the examples.

### DESCRIPTION OF THE FIGURES

FIG. 1: Old process for purifying Protein 1.

FIG. 2: Clapture process for protein 1.

FIG. 3: static capacity of different CEX resins for Protein 1, after 1 hour of stirring.

FIG. 4: Old process for purifying Protein 2.

FIG. 5: Clapture process for protein 2.

FIG. 6: static capacity of different CEX resins for Protein 2, after 1 hour of stirring.

### EXAMPLES

### Material

Protein 1 is a growth factor produced in insoluble bodies from E. coli. It has a molecular weight of 20 kDa and a pI of 10.5.

Protein 2: is a protein produced as a secreted protein in Pichia pastoris. It has a molecular weight of 40.1 kDa, and a pI of 5.85.

Hollow fibre:

| | | Size | Thres- | | Feature of the |
|---|---|---|---|---|---|
| Reference | Providers | lumen | hold | Surface | fibres |
| CFP-2-E-4MA | GE Healthcare | 1 mm | 0.2 μm | 420 cm² | PES |
| S02-E750-10-N | Spectrum | 1 mm | 750 kDa | 490 cm² | mPES |
| | | | | | (modified |
| | | | | | PES) |
| D02-E20U-10-N | Spectrum | 1 mm | 0.22 μm | 75 cm² | mPES |
| | | | | | (modified |
| | | | | | PES) |
| C06-E500-10-N | Spectrum | 1 mm | 750 kDa | 41 cm² | mPES |
| | | | | | (modified |
| | | | | | PES) |

Chromatographic resins

| Resins | ligands | Providers | Beads size | | Reference | Pores size |
|---|---|---|---|---|---|---|
| Toyopearl | CEX | Tosoh | 75 | μm | 21946 | 1000A |
| GigaCap CM-650M | | | | | | |
| Toyopearl | CEX | Tosoh | 75 | μm | 21833 | 1000A |
| GigaCap S-650M | | | | | | |
| SP Sepharose FF | CEX | GE Healthcare | 90 | μm | 17-0729-05 | — |
| Eschmuno CPS | CEX | Merck Millipore | 50 | μm | 1.20083 | — |
| Poros 50 HS | CEX | Thermo Fisher | 50 | μm | 1335911 | 500-1000A |
| Fractogel S03- | CEX | Merck Millipore | 20-40 | μm | 1.16890.0100 | 800A |

### Example 1: Old Purification Process for Protein 1

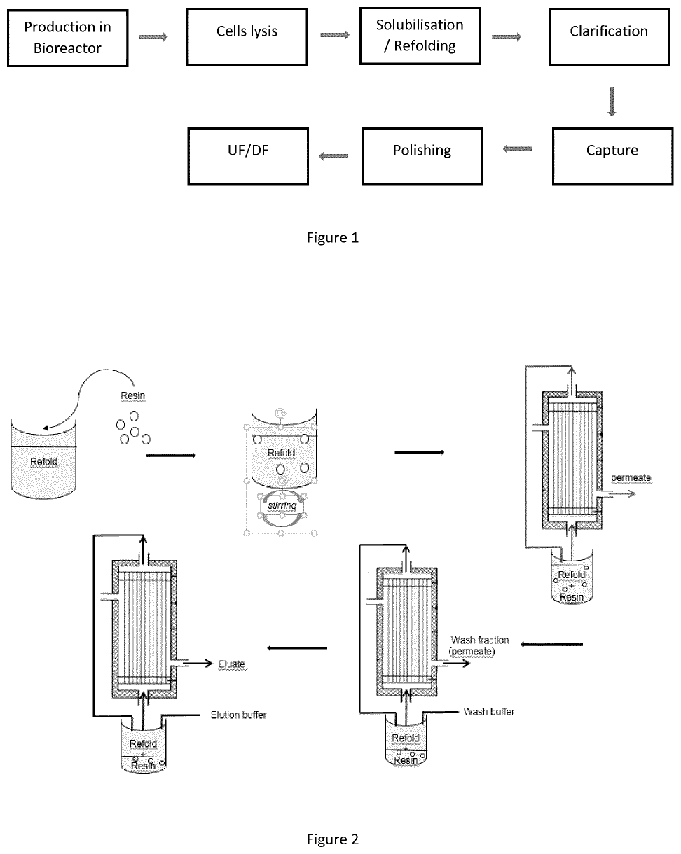

The old process for purifying Protein 1 comprised, after fermentation of recombinant E. coli cells in a bioreactor, the following steps (see FIG. 1):
a) Lysis of the cells contained in the crude sample comprising Protein 1 in order to release the inclusion bodies, as per routine procedure.
b) Solubilisation of the inclusion bodies and refolding of Protein 1 contained in the inclusion bodies, as per routine procedure, leading to a refold sample (quantity: 2,500 L).
c) Clarification of the refold sample on a Polyvinylidene Fluoride (PVDF; surface of 10-12 m², filterability of <300 L/m². The duration of this step was of about 120-180 min.
d) Capture of the protein of interest comprised in the pre-treated sample on a CEX resin, SP-SFF type having a loading capacity of 15 g/L, in bind elute mode, leading to an eluate comprising the protein of interest.
e) Polishing of protein 1 comprised in the eluate (i.e. further purification of protein 1).
f) UF/DF for final purification and concentration of the protein of interest.

According to the old process, the clarification step followed by the capture step with an SP-SFF resin on a chromatographic column had a duration of about 24 hours (about 5 hours for clarification and about 19 hours for capture). The yield was of 60% and the HCPs were below 250 ppm after the capture step. Purity of Protein 1 was 100%

### Example 2: New Purification Process for Protein 1

### Evaluation of the Filterability of a Hollow Fibre for Protein 1

First of all, it was necessary to check that hollow fibres could be used for Protein 1. Hollow fibres (PES) filterability was thus compared to PVDF filterability.

The conditions of filtration that were used are reported in the Table below (Table 1):

| | Hollow fibres | | PVDF | |
|---|---|---|---|---|
| Sample to be filtered | Refold sample | | Refold sample | |
| Cut-off properties | 0.22 | μ | 0.22 | μ |
| Exchange surface | 420 | cm² | 1000 | cm² |
| Recirculation flow rate | 2.4 | L/min | 2.4 | L/min |
| Filtrated volume | 15.8 | L | 15.8 | L |
| Average pressure | 0.138 | bars | 0.225 | bars |
| Filtration duration | 36 | min | 90 | min |

As shown in the above Table 1, it was possible to reduce the filtration duration by about 60%. Not only the filtration was faster with a hollow fibre but it was also performed: 1) at a lower pressure, decreasing the risks of clogging that can be observed frequently with membrane filters and 2) with smaller surface (420 cm² vs 1000 cm²), thereby reducing the needed filtration surface by about 60%. The use of hollow fibres did not impact the quality of the purified product (here Protein 1), as evidenced in Table 2 below:

| | Hollow fibres | PVDF |
|---|---|---|
| Yield (by RP-HPLC) | 100% | 100% |
| HCP (ppm) | 15 | 16 |
| SE-HPLC (% purity) | 99.85 | 99.84 |

### Evaluation of the Impact of Resin Beads on Hollow Fibres

It was needed to evaluate the impact of the resin beads on the hollow fibres as they could have an abrasive effect, leading to fibres deterioration. A study was thus performed with the aim to measure the water permeate flow rate. A constant flow rate over time would mean that the fibre is not impacted by the presence of beads. On the contrary if the flow rate is increased, it would be a sign of deterioration of the fibre. The conditions were as follow (Table 3):

| | Exchange | Lumen | | Recirculation |
|---|---|---|---|---|
| Cut-off | surface | size | Resin | flow rate |
| 750 kDa | 490 cm² | 1 mm | SP-SFF | 2.4 L/min |

A first water permeate flow rate was determined before addition of the resin beads in the system containing the hollow fibre: the baseline flow rate was of 300 LMHB. Then, SP-SFF beads were recirculated in a hollow fibre module. After 1 hour of recirculation, the permeate flow rate was measured. The test was repeated in 9 independent experiments. No negative effect of the beads on the hollow fibre was identified (data not shown).

### New Clapture Step

As hollow fibres can be efficiently used to filtrate Protein 1 and as resin beads do not damage the filtration membrane, clapture approach can be tested.

Preparation of the resin beads: before being used, the resin beads were washed one time with a buffer at high salth concentration (2M NaCl) in order to remove storage buffer from resin beads. Then, after centrifugation, supernatant was removed and an equilibration buffer (containing 50 mM Tris, 120 mM NaCl at pH 8.0) was added (volume added was equivalent to a minimum of 10 volume of resin). The equilibration of the beads was performed 3 times (with the same equilibration buffer). To check if the resin beads were properly equilibrated, pH and conductivity were measured in the last supernatant. The equilibration of the beads was good if the pH and the conductivity of the last supernatant correspond to the pH and the conductivity of the equilibration buffer.

The main steps of the clapture approach were the following (FIG. 2):
a) The equilibrated resin beads were added directly in the refold sample before any filtration steps. At this stage the refold sample has a pH of 8.0±0.05 and a conductivity of 16.5±0.5 mS/cm. The resin beads were not packed in a chromatographic column.
b) The mix resin beads+refold sample was stirred in order Protein 1 binds to the resin beads. The time of contact tested was 15 min to simulate the residence time of the old process.
c) The mix resin beads/Protein 1+refold sample was concentrated via filtration by hollow fibre.
d) The resin beads were washed via dialysis with a wash buffer (similar to the equilibration buffer, i.e. 50 mM Tris, 120 mM NaCl at pH 8.0) with the aim to remove the proteins and the impurities not bounds to the resin beads (which are eliminated with the permeate). The resin beads are in the retentate.
e) The washed resin beads (i.e. retentate) were subjected to elution buffer (containing 50 mM Tris, 1M NaCl at pH 8.0) in order to elute the Protein 1 of interest from the resin beads.

The recovery was only 15% vs 60% with old process. It was first needed to determine the best stirring time. In unpacked condition, the binding between protein and bead is different.

### Evaluation of Different Stirring Time, Resin and Capacity

As the recovery was very low for the first trial, stirring time parameter was evaluated in 3 points. Different CEX resin were tested at 2 capacities: 15 and 30 g/L (Before use, the resin beads were prepared as above mentioned).
a) The equilibrated resin beads were added directly in the refold sample before any filtration steps. At this stage the refold sample has a pH of 8.0±0.05 and a conductivity of 16.5±0.5 mS/cm. The resin beads were not packed in a chromatographic column.
b) Different CEX resins were tested: SP-SFF (the resin of the original process), Eschmuno CPS, POROS XS, Fractogel SO₃⁻, Giga Cap C650M and Giga Cap S650M.
c) The mix resin beads+refold sample was stirred in order Protein 1 binds to the resin beads. Different contact time were tested: 1 hour, 6 hours and 15 hours. Some samples were removed before next step in order to assess the binding capacity of each resin for Protein 1.
d) The mix resin beads/Protein 1+refold sample was centrifuged.
e) The supernatant was analysed.

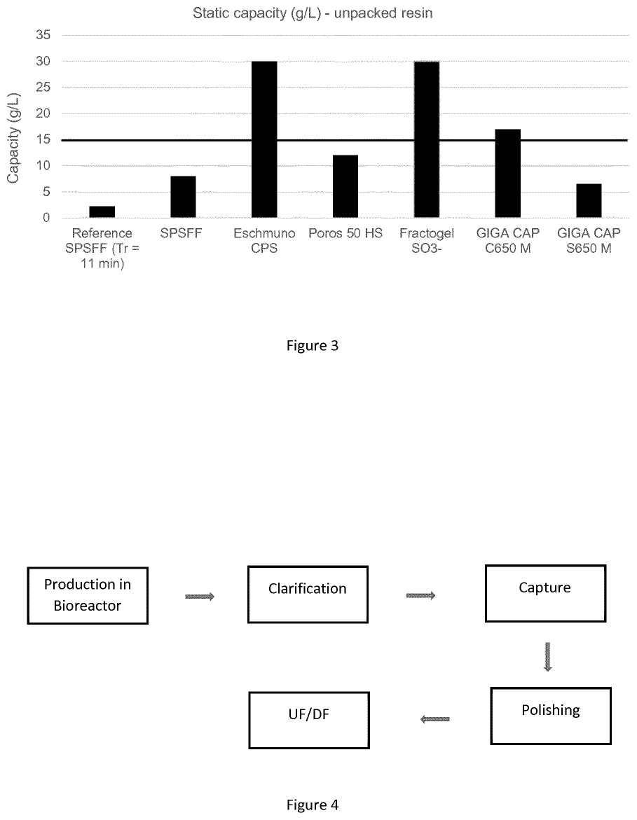

It was first needed to determine the best conditions of resin beads+stirring time. It was surprisingly found that Eschmuno CPS and Fractogel SO₃⁻, had capture capacity much higher than the other resins, as more than 30 g/L of Protein 1 could be captured after 1 hour of contact time (see FIG. 3).

### Confirmation of Process Conditions

a) The resin beads (Fractogel SO3−, equilibrated according to the same protocol as above) were added directly in the refold sample (at a capacity of 30 g/L of refold) before any filtration steps. At this stage the refold sample had a pH of 8.0±0.05 and a conductivity of 16.5±0.5 mS/cm. The resin beads were not packed in a chromatographic column.
b) The mix resin beads+refold sample was stirred in order Protein 1 binds to the resin beads. The time contact was 1 h.
c) The mix resin beads/Protein 1+refold sample was concentrated via filtration by hollow fibre (12 fold).
d) The resin beads were washed via dialysis with a wash buffer (similar to the equilibration buffer, i.e. 50 mM Tris, 120 mM NaCl at pH 8.0) with the aim to remove the proteins and the impurities not bounds to the resin beads (which are eliminated with the permeate). The resin beads are in the retentate
e) The washed resin beads (i.e. retentate) were adjusted at 1M NaCl to perform elution.
f) The retentate were dialyzed with elution buffer to elute the Protein 1 of interest from the resin beads.

The yield was at 46% and HCP were at 130 ppm.

After various testing, it was determined that for Protein 1, the best conditions were as follow:
Equilibration buffer containing 120 mM NaCl, 50 mM tris, pH 8.0
Step c): 12-fold concentration
Step d): wash buffer containing 120 mM NaCl, 50 mM tris, pH 8.0
Step e) adjustment of the retentate at 1M NaCl by addition of NaCl in powder followed by
f) elution with an elution buffer comprising 1M NaCl, 50 mM tris pH 8.0

According to the new process, the clarification step followed by the capture step with Fractogel 503 had a duration of 5 hour (including 1 hour of contact in step b)). The yield was of 46% and the HCPs were below 250 ppm after the capture step. Furthermore, with the original process, a big surface of membrane is necessary due to the fast fouling of the filter. With the new invention presented here, the form of hollow fibre as cylinder allowed to avoid clogging. Thus, the purification can be faster and used a smaller membrane.

Thanks to this new clapture step (in particular linked to short duration, simpler implementation, less resin needed, no need of packing the resin in a column), the costs were significantly reduced (data calculate for manufacturing scale) by about 65% for 1 run.

### Example 3: Old Purification Process for Protein 2

The old process for purifying Protein 2 comprised, after fermentation of recombinant P. pastoris in a bioreactor, the following steps (see FIG. 4):
a) Clarification of the harvest on a hollow fibres.
b) Capture of the protein of interest comprised in the pre-treated sample on a Mixed-mode resin, MEP type having a loading capacity of 50 g/L, in bind elute mode, leading to an eluate comprising the protein of interest.
c) Polishing of the protein of interest comprised in the eluate (i.e. further purification of the protein of interest).
d) UF/DF for final purification and concentration of the protein of interest.

According to the old process, the clarification step followed by the capture step with a MEP resin on a chromatographic column had a duration of about 18 hours (about 5 hours for clarification and about 13 hours for capture). The yield was of 95% and the HCPs were roughly 500 to 800 ppm after the capture step. Purity of Protein 2 was of 97.7%.

### Example 4: New Purification Process for Protein 2 (See FIG. 5)

### Evaluation of Different Resin

a) The resin beads (equilibrated as described in example 2) were added directly in the crude sample (100 g/L of crude sample) before any filtration steps. At this stage the crude sample has a pH of 7.0±0.1 and a conductivity of 4±0.5 mS/cm. The resin beads were not packed in a chromatographic column.
b) Different CEX resins were tested: SP-SFF, Eschmuno CPS, POROS 50HS, Fractogel SO₃⁻, Giga Cap C650M and Giga Cap S650M.
c) The mix resin beads+crude sample was stirred in order Protein 2 binds to the resin beads. The contact time tested is 1 hour.
d) The mix resin beads/Protein 2+crude sample was centrifuged.
e) The supernatant was analysed.

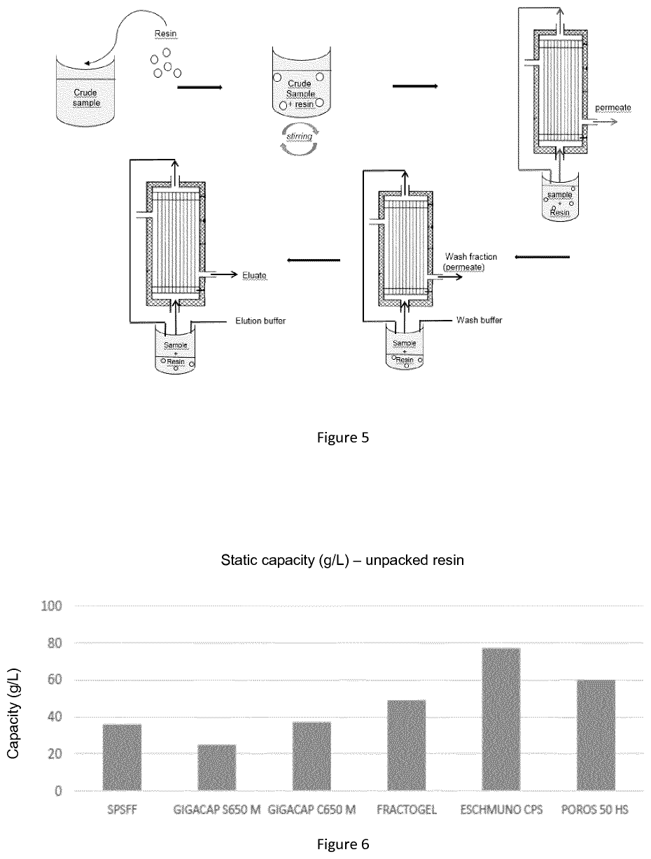

It was first needed to determine the best conditions of resin beads. It was surprisingly found that Eschmuno CPS and to a lesser extend POROS 50 HS, had capture capacity much higher than the other resins, as more than 80 g/L of Protein 2 could be captured after 1 hour of contact time with Eschmuno CPS and up to 60 g/L with POROS 50HS (see FIG. 6).

### Confirmation of Process Conditions

a) The resin beads (Eschmuno CPS, equilibrated as per the above protocol, except for the equilibration buffer) were added directly in the crude sample (at a capacity of 80 g/L) of crude sample before any filtration steps. In order to bind the molecule of interest to the resin, the sample was modified. The pH of the sample was adjusted at 4.0±0.2 with acetate. The resin beads were not packed in a chromatographic column.
b) The mix resin beads+refold sample was stirred in order Protein 2 binds to the resin beads. The time contact is 1 h.
c) The mix resin beads/Protein 2+crude sample was concentrated via filtration by hollow fibre.
d) The resin beads were washed via dialysis with a wash buffer with the aim to remove the proteins and the impurities not bounds to the resin beads (which are eliminated with the permeate). The resin beads are in the retentate.
e) The retentate were dialyzed with elution buffer (200 mM Tris, pH 11) to elute the Protein 2 of interest from the resin beads.

After various testing, it was determined that for Protein 2, the best conditions were as follow:
Equilibration buffer: 50 mM acetate pH 4.0.
Step c): 1.8-fold concentration.
Step d): wash buffer was similar to the equilibration buffer but with a pH slightly above (while being still below the pI of the protein to be purified).
Step e) elution with an elution buffer comprising 200 mM Tris, pH11.

According to the new process, the clapture step with Eschmuno CPS had a duration of 5 hour (including 1 hour of contact in step b)). The yield was of 100% and the HCPs were 35 000 ppm after the capture step.

Thanks to this new clapture step, and in particular linked to short duration, simpler implementation, less resin needed, no need of packing the resin in a column, the costs were significantly reduced by about 64% at manufacturing scale (for 1 run).

### Overall Conclusions:

The inventors surprisingly found that the new process called clapture decreases the purification time (clapture decreases by at least a factor 3 the time for the first step of purification process compared to a process involving both clarification and a first purification step at small scale) as well as decreases the cost of production by about 65% (for one run) for the purification of proteins produced either in E. coli or in Pichia.

### REFERENCES

1. WO9747650
2. WO0048703
3. Sambrook et al., 1989, and updates

1-14. (canceled)

15. A method for purifying a polypeptide of interest from a sample containing said polypeptide of interest and impurities, said process comprising the steps of:
i) contacting the sample containing the polypeptide of interest and impurities with a chromatography resin, without submitting said sample to an initial clarification step;
ii) incubating the sample from step i) with the chromatography resin for a sufficient time to allow the resin to bind the polypeptide of interest;
iii) recirculating the chromatography resin in hollow fibres or a tangential filtration system, thereby concentrating the polypeptide of interest while removing the impurities;
iv) washing by diafiltration the sample containing the polypeptide of interest and the impurities in order to remove impurities;
v) eluting the polypeptide of interest from the chromatography resin; and
vi) recovering the purified polypeptide of interest from the chromatography resin by diafiltration.

16. The method according to claim 15, wherein the chromatography resin is selected from the group consisting of protein A, protein A related, cation-exchange and anion-exchange resins.

17. The method according to claim 16, wherein the chromatography resin is a cation-exchange resin.

18. The method according to claim 15, wherein the recovered purified polypeptide is further purified through at least one additional purification step.

19. The method according to claim 18, wherein the at least one additional purification step is selected from the group consisting of affinity chromatography, cation exchange chromatography, anion exchange chromatography and mixed mode chromatography.

20. The method according to claim 18, wherein the recovered purified polypeptide is optionally further concentrated using ultrafiltration (UF), diafiltration (DF) or a combination thereof (UF/DF).

21. The method according to claim 15, wherein the pH of the sample containing the protein of interest is at least 1 unit higher than the pI of the protein of interest.

22. The method according to claim 15, wherein the sample containing the protein of interest to be purified is at a conductivity in the range of about 0 to about 20 mS/cm.

23. The method according to claim 15, wherein the sample containing said polypeptide of interest and impurities is a harvest fluid or a post-harvest.

24. The method according claim 23, wherein the harvest fluid is a crude harvest, a crude post-harvest, a harvest, or a post-harvest submitted to solubilization and refolding.

25. The method according to claim 15, wherein the protein of interest has been produced in a recombinant cell and is either secreted by the recombinant cell or is contained in inclusion bodies produced by the recombinant cell.

26. The method according to claim 25, wherein the recombinant cell is a prokaryotic cell or a lower eukaryotic cell.

27. The method according to claim 25, wherein the recombinant cell is a yeast cell or a bacterial cell.

28. The method according to claim 15, wherein the polypeptide of interest is selected from the group consisting of a recombinant protein, a fusion protein, an immunoglobulin, an antibody, and fragments thereof.

29. The method according to claim 15, wherein the impurities are selected from the group consisting of aggregates or fragments, or mixtures thereof, of the protein of interest, one or more of host cell proteins, endotoxins, viruses, nucleic acid molecules, lipids, polysaccharides, and combinations thereof.

30. The method according to claim 15, wherein the step of incubating the sample from step i) with the chromatography resin for a sufficient time to allow the resin to bind the polypeptide of interest is performed under stirring conditions.
